(19) United States
(12) Patent Application Publication
(10) Pub. No.: US 2025/0211126 A1
(43) Pub. Date: Jun. 26, 2025

(54) POWER SUPPLY SYSTEM FOR SURGICAL ROBOT

(71) Applicant: Precision Robotics (Hong Kong) Limited, Shatin, N.T. (HK)

(72) Inventors: Ping Lai Benny Lo, Shatin, N.T. (HK); Ka King Wong, Shatin, N.T. (HK)

(21) Appl. No.: 18/690,634

(22) PCT Filed: Aug. 17, 2023
(86) PCT No.: PCT/CN2023/113635

§ 371 Date: Mar. 8, 2024

Related U.S. Application Data
(60) Provisional application No. 63/398,550, filed on Aug. 17, 2022.

(51) Int. Cl. H02M 7/00; A61B 34/30; H02M 7/04

(52) U.S. Cl. CPC H02M 7/003; H02M 7/04; A61B 34/30

(57) ABSTRACT

The present disclosure provides a power supply system for a surgical robot, which includes an AC to DC converter module, a power supply interface board and at least one DC power control board. The AC to DC converter module is configured to convert obtained external AC power into DC power and output the DC power to the power supply interface board. The DC power control board is configured to obtain the DC power from the power supply interface board and supply power to the surgical robot through its own output interface. According to the present disclosure, there is no need to set an inverter in the whole power supply system, thus simplifying the structure of the power on the premise of ensuring the power supply requirements, avoiding the circuit loss caused thereby, and being beneficial to expanding the applicable scenarios of the surgical robot.

### CROSS-REFERENCE TO RELATED APPLICATIONS

This Application is a National Stage Application under 35 U.S.C. § 371 of PCT Application No. PCT/CN2023/113635, filed Aug. 17, 2023, which claims priority to U.S. Provisional Patent Application No. 63/398,550, filed Aug. 17, 2022, the entire contents of which are incorporated by reference herein.

### TECHNICAL FIELD

The present disclosure relates to the technical field of a surgical robot, in particular to a power supply system for a surgical robot.

### BACKGROUND

With the popularity of the robotic surgery system, more and more surgical operations are realized by surgical robots. However, when designing a surgical robot, it is often necessary to consider different application environments and instruments adapted to them, which leads to more complicated power supply system. A traditional power supply system of a surgical robot usually uses multiple local transformers to provide direct current (DC) power to each component. However, due to the need to configure multiple inverters, the power supply system is easy to be bulky, the power supply management is complicated, and the system conversion loss is also large. In addition, the current power supply system for surgical robots often can't give consideration to the matching of the output of alternating current (AC) power and the DC power with external products, which will affect and limit the application of surgical robots.

### SUMMARY

A technical problem to be solved by the present disclosure is to provide a power supply system for a surgical robot for overcoming the above defects in the related art, which includes an AC to DC converter module, a power supply interface board and at least one DC power control board.

The AC to DC converter module and the DC power control board are respectively electrically connected to the power supply interface board through correspondingly arranged interfaces.

The AC to DC converter module is configured to convert obtained external AC power into DC power and output the DC power to the power supply interface board. The DC power control board is configured to obtain the DC power from the power supply interface board and supply power to the surgical robot through its own output interface.

Preferably, the power supply system for the surgical robot further includes a DC to DC converter module.

The DC to DC converter module is electrically connected to the AC to DC converter module and the power supply interface board respectively.

The AC to DC converter module directly outputs first DC power to the power supply interface board, and further outputs a second DC further to the power supply interface board through the DC to DC converter module.

The power supply interface board provides the first DC power or the second DC power to the corresponding DC power control board respectively.

This solution addresses the diversity requirements of output devices in the power supply system. By providing the DC to DC converter module, the power supply system not only provides a DC input directly from the AC to DC converter module to the power supply interface board, but also provides another DC input in parallel, thus enriching the output parameter selection of the power supply interface board. At the same time, the DC to DC converter module may also directly supply power to appropriate devices simultaneously, which is conducive to increasing the application scenarios of surgical robots and is better compatible with some surgical scenarios with many devices.

Preferably, the power supply system for the surgical robot further includes a first DC power control board and a second DC power control board.

The first DC power control board and the second DC power control board respectively obtain the first DC power and the second DC power from the power supply interface board through correspondingly arranged interfaces, and provide the first DC power and the second DC power to the surgical robot through their own output interfaces.

In this solution, two DC power control boards are arranged for power supply modes of different gears of DC power, and complex power supply requirements of surgical robots are met by providing two DC power control boards corresponding to different output currents. At the same time, other DC power control boards may be added and their corresponding power output parameters may be changed according to the needs of actual environment and different device.

Preferably, the first DC power control board includes a first output interface, a second output interface, a third output interface, a fourth output interface, a fifth output interface and a sixth output interface. The second DC power control board includes a seventh output interface, an eighth output interface, a ninth output interface, a tenth output interface, an eleventh output interface and a twelfth output interface.

Positive temperature coefficients corresponding to the first output interface, the second output interface, the third output interface, the fourth output interface, the fifth output interface and the sixth output interface are 5 amps/60 volts, 3.75 amps/60 volts, 3.75 amps/60 volts, 5 amps/60 volts, 3.75 amps/60 volts and 3.75 amps/60 volts, respectively. Positive temperature coefficients of the seventh output interface, the eighth output interface, the ninth output interface, the tenth output interface, the eleventh output interface and the twelfth output interface are 5 amps/30 volts, 4 amps/30 volts, 4 amps/30 volts, 5 amps/30 volts, 4 amps/30 volts and 4 amps/30 volts, respectively.

By matching the corresponding positive temperature coefficient to different output interfaces, this solution can meet the usual power supply requirements of surgical robots, such as the power supply of a mechanical arm of the surgical robot. In addition, some mainstream special power supply requirements such as snake robots can also be met.

Preferably, the first DC power control board and the second DC power control board respectively include a current sensor, a voltage sensor, a temperature sensor and a circuit protection component arranged corresponding to the output interfaces.

Preferably, the DC to DC converter module is further provided with the second DC power directly outputted.

Preferably, the power supply system for the surgical robot further includes an AC power control board.

The external AC power is inputted to the AC power control board, so that the power supply system outputs AC power. The external AC power is inputted into the AC to DC converter module, so that the power supply system outputs DC power.

This solution achieves that the conversion is performed by the AC to DC converter module, and at the same time, the AC power directly outputted can also make the output of the entire power supply system more abundant and reasonable, meeting the needs of different electrical devices.

Preferably, the power supply system for the surgical robot further includes a power filter, which is configured to carry out an electromagnetic finishing treatment on the external AC power. In this solution, the input power is stably controlled by providing a power filter, which is beneficial to form a more stable working environment of the power supply system and provide a more stable and reliable power output.

The power filter is electrically connected to the AC power control board and the AC to DC converter module respectively.

Preferably, the AC to DC converter module is a power supply unit.

Preferably, the power supply interface board is provided with a plurality of interfaces for directly outputting a power supply.

The positive progress effect of the present disclosure is in that: by providing the power supply system for surgical robots in the present disclosure, the AC power input in the power supply system is realized based on providing the AC to DC converter module, the power supply interface board and the DC power module, so that the DC power can be directly obtained by all related system components, and there is no need to set an inverter in the whole power supply system, thus simplifying the structure of the power on the premise of ensuring the power supply requirements, avoiding the circuit loss caused thereby, optimizing the hardware structure and control logic of the power supply system, and being beneficial to expanding the applicable scenarios of the surgical robot.

### BRIEF DESCRIPTION OF THE DRAWINGS

FIG. 1 is a schematic diagram of modules of a power supply system for a surgical robot in Embodiment 1 of the present disclosure.

FIG. 2 is a schematic diagram of setup of an interface of a DC power control board of the power supply system for the surgical robot in Embodiment 1 of the present disclosure.

FIG. 3 is a schematic diagram of a structure of the DC power control board of the power supply system for the surgical robot in Embodiment 1 of the present disclosure.

FIG. 4 is a schematic diagram of an AC power control board of the power supply system for the surgical robot in Embodiment 1 of the present disclosure.

### DETAILED DESCRIPTION

In order to explain the technical solution of the embodiments of this specification more clearly, the accompanying drawings needed in the description of the embodiments will be briefly introduced below. Obviously, the accompanying drawings in the following description are only some examples or embodiments of this specification. For those skilled in the art, this specification can be applied to other similar situations according to these accompanying drawings without creative work. Unless it is obvious from the linguistic context or otherwise stated, the same reference signs in the accompanying drawings represent the same structure or operation.

As shown in this specification, the words “a”, “an”, “one” and/or “the” do not refer to the singular, but may also include the plural. Generally, the terms “include” and “contain” only imply the inclusion of clearly identified steps and elements, and these steps and elements do not constitute an exclusive list, and a method or device may also contain other steps or elements.

### Embodiment 1

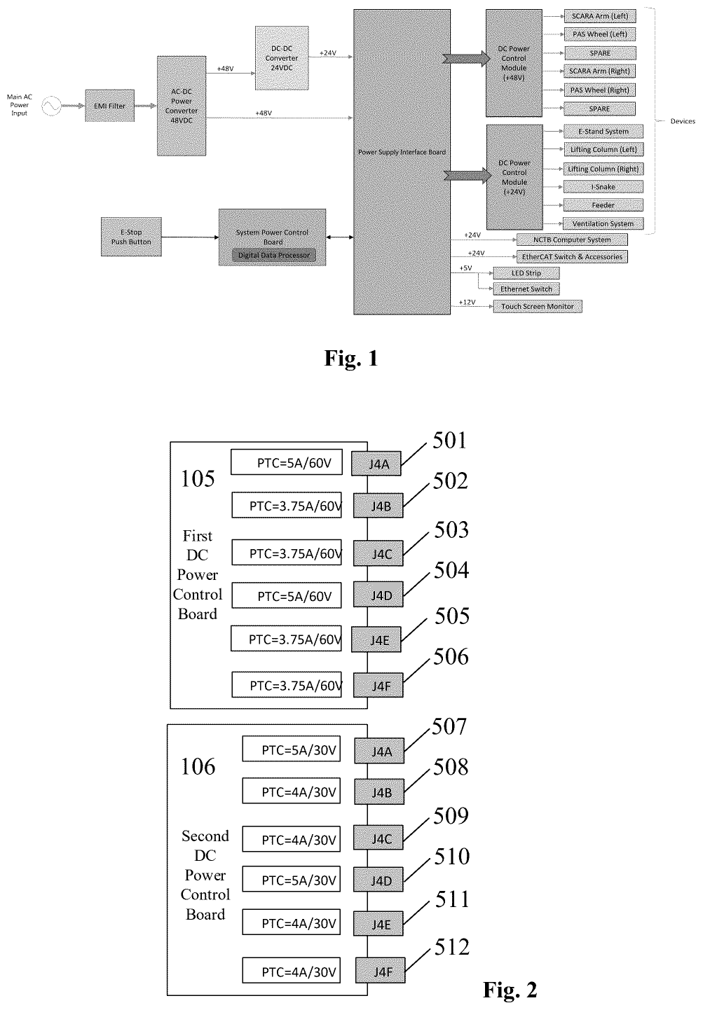

As shown in FIG. 1, this embodiment specifically provides a power supply system for a surgical robot, including an AC to DC converter module 101, a power supply interface board 102 and at least one DC power control board. The AC to DC converter module 101 and the DC power control board are respectively electrically connected to the power supply interface board 102 through correspondingly arranged interfaces. The AC to DC converter module 101 is configured to convert obtained external AC power into DC power and output the DC power to the power supply interface board 102. Preferably, the AC to DC converter module may be a power supply unit (PSU). The DC power control board is configured to obtain the DC power from the power supply interface board 102 and supply power to the surgical robot through its own output interface. Those skilled in the art can understand that the power supply interface board 102 may be a field programmable gate array (FPGA) board. The DC power control board may be electrically connected to the power supply interface board 102 through a corresponding interface, or may be connected by other means.

Preferably, the power supply interface board 102 may also be provided with several interfaces for directly outputting power supply, such as being connected to a display device, etc. The layout of the interfaces may be uniformly designed in combination with the number of interfaces of the DC power control board and the size of the power supply interface board 102, for example, the interface for directly outputting power supply is arranged on the other side of the DC power control board.

As a preferred embodiment, the power supply system for the surgical robot further includes a DC to DC converter module 103. The DC to DC converter module 103 is electrically connected to the AC to DC converter module 101 and the power supply interface board 102, respectively. The AC to DC converter module 101 directly outputs first DC power to the power supply interface board 102, and further outputs second DC power to the power supply interface board 102 through the DC to DC converter module 103. The power supply interface board 102 provides 48V DC power or 24V DC power to the corresponding DC power control board, respectively. The first DC power and the second DC power in this embodiment are described by taking 48V DC power and 24V DC power as an example respectively, and those skilled in the art can understand that they do not constitute any restrictions on the present disclosure.

In this embodiment, by providing the DC to DC converter module 103, the power supply system not only provides a DC input directly from the AC to DC converter module to the power supply interface board, but also provides another DC input in parallel, thus enriching the output parameter selection of the power supply interface board. At the same time, the DC to DC converter module 103 itself can also directly supply power to the appropriate devices simultaneously, which is beneficial to the adaptability of the intraoperative environment and is better compatible with some scenarios with many operating room devices.

As a preferred embodiment, the power supply system for the surgical robot further includes a first DC power control board 105 and a second DC power control board 106. The first DC power control board 105 and the second DC power control board 106 respectively obtain the 48V DC power and the 24V DC power from the power supply interface board 102 through correspondingly arranged interfaces, and provide the 48V DC power and the 24V DC power to the surgical robot through their own output interfaces.

In this embodiment, two DC power control boards corresponding to different output currents are provided to meet the complicated power supply requirements of the surgical robot. Of course, as mentioned above, the 48V and 24V DC power are just examples herein. According to the needs of the actual environment and different devices, other DC power control boards may be added or output parameters of their corresponding power may be changed.

As shown in FIG. 2, as a preferred embodiment, the first DC power control board 105 includes a first output interface 501, a second output interface 502, a third output interface 503, a fourth output interface 504, a fifth output interface 505 and a sixth output interface 506. The second DC power control board 106 includes a seventh output interface 507, an eighth output interface 508, a ninth output interface 509, a tenth output interface 510, an eleventh output interface 511 and a twelfth output interface 512.

Positive temperature coefficients corresponding to the first output interface 501, the second output interface 502, the third output interface 503, the fourth output interface 504, the fifth output interface 505 and the sixth output interface 506 are 5 amps/60 volts, 3.75 amps/60 volts, 3.75 amps/60 volts, 5 amps/60 volts, 3.75 amps/60 volts, respectively. Positive temperature coefficients of the seventh output interface 507, the eighth output interface 508, the ninth output interface 509, the tenth output interface 510, the eleventh output interface 511 and the twelfth output interface 512 are 5 amps/30 volts, 4 amps/30 volts, 4 amps/30 volts, 5 amps/30 volts, 4 amps/30 volts, respectively. It can be understood that the corresponding relationship between the above-mentioned output interfaces and their positive temperature coefficients does not limit the concept of the present disclosure, and other parameters suitable for surgical robot components, including but not limited to the number of output interfaces and their corresponding positive temperature coefficients, may be set as needed.

In this embodiment, by matching the corresponding positive temperature coefficients to different output interfaces, the usual power supply requirements of surgical robots, such as the power supply of the mechanical arm of the surgical robot, can be met. In addition, some mainstream special power supply requirements such as snake robots can also be met. Further, after receiving the power supply demand information, parameters of each interface can be automatically adjusted and matched, that is, adaptive, or indication information can be sent according to the existing parameters, for example, the user is prompted on the display screen which devices the interface is suitable for; or send out alarm information, such as reminding users which devices the interface is not suitable for accessing.

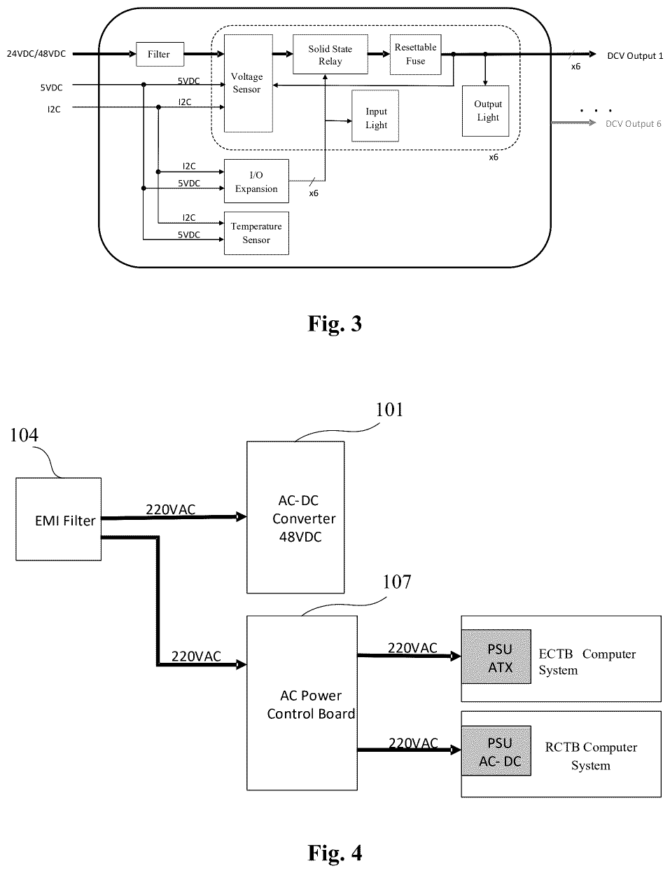

As a preferred embodiment, the first DC power control board 105 and the second DC power control board 106 respectively include a current sensor, a voltage sensor, a temperature sensor and a circuit protection component arranged corresponding to the output interfaces. As shown in FIG. 3, the circuit protection component may be a resettable fuse, and at the same time, a plurality of groups of the same or similar DC power control boards may be provided to form a DC output that meets the needs. In this embodiment, the DC power control board is provided with a monitoring module such as a voltage sensor and a circuit protection component such as a resettable fuse, which can form an effective monitoring and repair mechanism for the DC power control board which is one of the core components of the power supply system, ensuring that the DC power control board can provide a stable DC power output and provide safe and stable output supply for the electrical device.

As a preferred embodiment, the DC to DC converter module 103 is further provided with 24V DC power directly outputted. The output of the DC to DC converter module may be connected to an application computer with a corresponding board, such as a neurobehavioral core test battery (NCTB) host system, and of course, the application computer may also be other devices that meet the corresponding DC input parameters.

Referring to FIG. 4, as a preferred embodiment, the power supply system for the surgical robot further includes an AC power control board 107. The external AC power is inputted to the AC power control board 107, so that the power supply system outputs AC power. At the same time, the external AC power is inputted to the AC to DC converter module 101, so that the power supply system outputs DC power. Preferably, the power supply system for the surgical robot further includes a power filter 104, which is configured to carry out an electromagnetic finishing treatment on the external AC power. The power filter is electrically connected to the AC power control board and the AC to DC converter module 101 respectively.

By providing the power filter 104, the power inputted is stably controlled. While the AC power control board 107 performs conversion through the AC to DC converter module 101, it directly outputs AC power, which can make the output form of the whole power supply system more abundant and reasonable, and meet the requirements of different electrical devices.

The AC power input in the power supply system is realized by the power supply system for the surgical robot based on providing the AC to DC converter module, the power supply interface board and the DC power module, so that the DC power may be directly obtained by all related system components, and there is no need to set an inverter in the whole power supply system, thus simplifying the structure of the power on the premise of ensuring the power supply requirements, avoiding the circuit loss caused thereby, optimizing the hardware structure and control logic of the power supply system, and being beneficial to expanding the applicable scenarios of the surgical robot.

### Embodiment 2

This embodiment provides a power supply control method for the power supply system for the surgical robot in Embodiment 1. As mentioned above, the surgical robot includes a power control module for outputting power, and the control method includes:

S1, reading starting time sequence information of corresponding electrical devices of the surgical robot; and

S2, controlling the power control module to supply power to the electrical devices in turn, according to the starting time sequence information.

In step S1, corresponding starting time sequence information is obtained for different electrical devices, and the time sequence information may be expressed by relative time (such as the startup sequence of several devices), absolute time (such as turning on a device when it reaches a certain moment) or their combination. On this basis, in step S2, the power control module is controlled to power on each electrical device in turn according to the starting time sequence, so as to realize power consumption peak shifting and avoid a corresponding over-current phenomenon. Of course, several electrical devices involved in the starting time sequence may be started at the same time, and there is no restriction that they must have a sequence, as long as they meet the requirements of over-current protection. Preferably, when the surgical robot is started, step S1 is automatically executed to start the detection and control of power supply. The power supply system may firstly meet the working requirements of the corresponding DC and AC conversion modules, and the mechanical control system and related components may be powered on before the computer device.

When the power control module includes at least one DC power control board and at least one AC power control board. The electrical devices include a DC electrical device and an AC electrical device. The DC power control board is configured to output DC power to a DC electrical device, and the AC power control board is configured to output AC power to an AC electrical device. Preferably, step S2 includes:
controlling the AC power control board and the DC power control board to supply power to the DC electrical device and the AC electrical device in turn, respectively; where, the starting time sequence information is set according to working parameters of the DC electrical device and the AC electrical device, and the working parameters include at least one of starting current and rated current.

Specifically, when the power supply system is provided with the DC power output and the AC power output respectively, the types of the above-mentioned power outputs are distinguished and corresponding control is carried out in combination with the types of corresponding electrical device, so that the starting time sequence of the electrical device may be determined according to the working parameters such as the starting current and rated current, so as to ensure a stable output under the condition that the power supply system corresponds to a variety of different devices.

As a preferred embodiment, the DC electrical device includes mechanical components corresponding to the surgical robot. The AC electrical device includes a computer device corresponding to the surgical robot. In step S2, an order of supplying power to the DC electrical device and the AC electrical device in turn is to supply power to the computer device first, and then to corresponding electric equipment and motorized device. Specifically, a power transmission control is carried out for two types of electrical devices that are often involved in the application scenarios of surgical robots. First, power is supplied to the computer device, so that relevant control instructions are issued, and then power is supplied to the corresponding electric equipment and motorized device, so that various application requirements in the robot surgery environment are met.

The power supply control method for the surgical robot in this embodiment is based on the analysis of different electrical components of the surgical robot, and the corresponding electrical control logic is reasonably designed, so that the components of the surgical robot system can obtain independent DC power supply. By controlling the power-on time of the electrical components, the current surge can be effectively prevented when the electrical components are powered on, and the surgical robot can effectively work safely and stably, which has the significance of industry promotion.

### Embodiment 3

This embodiment further provides a computer-readable storage medium, on which a computer program is stored, and when executed by a processor, the computer program realizes the steps in the power supply control method for the surgical robot of the above embodiment. The readable storage medium may be a portable disk, a hard disk, a random access memory, a read-only memory, an erasable programmable read-only memory, an optical storage device, a magnetic storage device or any suitable combination of the above.

In a possible embodiment, the present disclosure may also be realized in the form of a program product, which includes a program code, and when the program product is run on a terminal device, the program code is used to make the terminal device execute the steps in the power supply control method for the surgical robot as described above. The program code for executing the present disclosure may be written in any combination of one or more programming languages, and the program may be completely executed on a user device, partially executed on the user device, executed as an independent software package, partially executed on the user device and partially executed on a remote device, or completely executed on the remote device.

Although specific embodiments of the present disclosure have been described above, those skilled in the art should understand that these are merely illustrative, and the scope of protection of the present disclosure is defined by the appended claims. Those skilled in the art may make many changes or modifications to these embodiments without departing from the principle and substance of the present disclosure, but these changes and modifications all fall within the protection scope of the present disclosure.

What is claimed is:

1. A power supply system for a surgical robot, comprising an alternating current (AC) to direct current (DC) power module, a power supply interface board and at least one DC power control board, wherein,
the AC to DC converter module and the DC power control board are respectively electrically connected to the power supply interface board through correspondingly arranged interfaces; and
the AC to DC converter module is configured to convert obtained external AC power into DC power and output the DC power to the power supply interface board; and the DC power control board is configured to obtain the DC power from the power supply interface board and supply power to the surgical robot through its own output interface.

2. The power supply system for the surgical robot according to claim 1, further comprising a DC to DC converter module, wherein,
the DC to DC converter module is electrically connected to the AC to DC converter module and the power supply interface board respectively;
the AC to DC converter module directly outputs first DC power to the power supply interface board, and further outputs second DC power to the power supply interface board through the DC to DC converter module; and
the power supply interface board provides the first DC power or the second DC power to the corresponding DC power control board respectively.

3. The power supply system for the surgical robot according to claim 2, further comprising a first DC power control board and a second DC power control board, wherein,
the first DC power control board and the second DC power control board respectively obtain the first DC power and the second DC power from the power supply interface board through correspondingly arranged interfaces, and provide the first DC power and the second DC power to the surgical robot through their own output interfaces respectively.

4. The power supply system for the surgical robot according to claim 3, wherein the first DC power control board comprises a first output interface, a second output interface, a third output interface, a fourth output interface, a fifth output interface and a sixth output interface; and the second DC power control board comprises a seventh output interface, an eighth output interface, a ninth output interface, a tenth output interface, an eleventh output interface and a twelfth output interface; and
positive temperature coefficients corresponding to the first output interface, the second output interface, the third output interface, the fourth output interface, the fifth output interface and the sixth output interface are 5 amps/60 volts, 3.75 amps/60 volts, 3.75 amps/60 volts, 5 amps/60 volts, 3.75 amps/60 volts and 3.75 amps/60 volts, respectively; and positive temperature coefficients of the seventh output interface, the eighth output interface, the ninth output interface, the tenth output interface, the eleventh output interface and the twelfth output interface are 5 amps/30 volts, 4 amps/30 volts, 4 amps/30 volts, 5 amps/30 volts, 4 amps/30 volts and 4 amps/30 volts, respectively.

5. The power supply system for the surgical robot according to claim 4, wherein the first DC power control board and the second DC power control board respectively comprise a current sensor, a voltage sensor, a temperature sensor and a circuit protection component arranged corresponding to the output interfaces.

6. The power supply system for the surgical robot according to claim 2, wherein the DC to DC converter module is further provided with the second DC power directly outputted.

7. The power supply system for the surgical robot according to claim 1, further comprising an AC power control board, wherein,
the external AC power is inputted to the AC power control board, so that the power supply system outputs AC power; and the external AC power is inputted to the AC to DC converter module, so that the power supply system outputs DC power.

8. The power supply system for the surgical robot according to claim 1, further comprising a power filter, which is configured to carry out an electromagnetic finishing treatment on the external AC power, wherein,
the power filter is electrically connected to the AC power control board and the AC to DC converter module respectively.

9. The power supply system for the surgical robot according to claim 1, wherein the AC to DC converter module is a power supply unit.

10. The power supply system for surgical robot according to claim 1, wherein the power supply interface board is provided with a plurality of interfaces for directly outputting a power supply.
